(19) United States
(12) Patent Application Publication
(10) Pub. No.: US 2026/0174593 A1
(43) Pub. Date: Jun. 25, 2026

(54) CONTROLLED EXOTHERMIC HEAT PACK

(71) Applicant: Hagay Weisbrod, Kibbutz Kinneret (IL)

(72) Inventor: Hagay Weisbrod, Kibbutz Kinneret (IL)

(21) Appl. No.: 19/543,900

(22) Filed: Feb. 19, 2026

Related U.S. Application Data
(63) Continuation-in-part of application No. PCT/IL2024/050853, filed on Aug. 25, 2024.
(60) Provisional application No. 63/534,832, filed on Aug. 27, 2023.

(51) Int. Cl. A61F 7/03; A61F 7/02; C09K 5/06; C09K 5/16

(52) U.S. Cl. CPC A61F 7/034; C09K 5/063; C09K 5/16; A61F 2007/0247; A61F 2007/0253; A61F 2007/036

(57) ABSTRACT

A system and method for controlling the surface temperature of a heat pack, wherein the heating bag comprises an exothermic chemical reaction as the heat source, a phase changing material as a buffer, and a means of controlling the rate of reaction. Optionally, the system may include one or more thermally conducting materials to increase the rate of heat transfer from the heat pack to the user.

### RELATED APPLICATIONS

This application is a Continuation in Part of International patent application PCT/IL2024/050853 filed on 25 Aug. 2024, which claims the benefit of priority under 35 USC § 119(e) of U.S. Provisional Patent Application No. 63/534,832 filed on 27 Aug. 2023 with the United States Patent and Trademark Office.

### FIELD AND BACKGROUND OF THE INVENTION

The present invention in some embodiments thereof relates to a system and method for controlling the surface temperature of a heat pack.

Heat packs are used as a heating source to treat hypothermia (e.g., due to cold exposure, for example, people lost/trapped in cold conditions), in hospitals to keep users who are anesthetized warm while transferring them between preparation rooms, operating room recovery rooms, etc. Additionally, heat packs are used for warming aching muscles, and provide heat therapy for muscle and joint pain, menstrual cramps, and arthritis, etc.

Some Heat packs include one or more reactive substances that are triggered when activated and/or when a seal is broken. For example, breaking the seal allows reactants to mix, thereby starting an exothermic reaction that creates heat over an extended period. In some examples, a heat pack can include two reactants that produce heat when they are mixed together. Alternatively, a heat pack may include one or more reactants that produce heat when exposed to air.

A problem is how to reach a desired temperature quickly and control the temperature. A heat pack for a human should provide a temperature ranging between about 37° C. to about 43° C. and/or between about 38° C. to about 42° C. (if the temperature is less than 37° C. the bag may add little or no heat to the body), but if the temperature on the skin rises to above about 43° C. for a significant time, it may cause scalding and/or serious injury and/or burns.

U.S. Pat. No. 8,431,387 appears to disclose exothermic and/or endothermic chemical reactions in combination with phase change materials which can produce output temperature(s) within strict tolerances without requiring expensive and complicated external equipment to generate and maintain an output temperature. Similarly, an exothermic phase change material, which generates heat as a consequence of crystallizing a supercooled liquid, can generate heat at a constant temperature, without requiring expensive and complicated external equipment, as a consequence of the liquid form of the exothermic phase change material being in equilibrium with the solid form of the exothermic phase change material. Numerous biological and chemical processes and/or diagnostic devices require a constant temperature or temperatures for set periods of time. An example completely non-instrumented diagnostic platform based on nucleic acid amplification is described, which is particularly suited for use in developing countries that may not have access to expensive and complicated external equipment.

U.S. Pat. No. 9,605,874 appears to disclose a heat pack including a housing, a first phase change material (PCM) that is contained in the housing, and a thermal storage buffer that is contained in the housing. The thermal storage buffer includes a second PCM. An initiator is in operative contact with the first PCM. Activation of the initiator causes crystallization of the first PCM, and the first PCM cooperates with the thermal storage buffer to provide heat at a predetermined temperature range upon crystallization of the first PCM.

Additional art includes, US 2007/0142882, U.S. Pat. Nos. 8,137,392, 6,099,556, 4,366,804, US 2004/0116990, US 2006/0178717, and U.S. Pat. No. 7,794,649 describe various means of adjusting heat produced by heating packs.

### SUMMARY OF THE INVENTION

According to an aspect of some embodiments of the invention, there is provided a heat pack including: a heat source including an exothermic reaction; a buffer including a phase transition material; and a thermally conducting material configured to conduct heat between the heat source to the buffer and an outer contact surface of the heat pack.

According to some embodiments of the invention, the exothermic reaction includes an iron oxidation reaction.

According to some embodiments of the invention, the phase changing material undergoes endothermic phase transition.

According to some embodiments of the invention, subsequent to the endothermic phase transition, the phase changing material undergoes exothermic phase transition.

According to some embodiments of the invention, the phase changing material undergoes a phase transition at a temperature ranging between 40° C. to about 45° C.

According to some embodiments of the invention, the phase transition is reversible.

According to some embodiments of the invention, the phase transition is selected from the group consisting of: melting, crystallization, fusion, freezing, evaporation, sublimation, or any combination thereof.

According to some embodiments of the invention, the thermally conducting material is configured to provide a path along which heat from the heat source is conducted directly to a body of a user.

According to some embodiments of the invention, the thermally conducting material includes a thermally conducting layer.

According to some embodiments of the invention, the thermally conducting layer covers a portion of an outer surface of the heat pack.

According to some embodiments of the invention, the thermally conducting layer forms an interface between the heat source and the buffer.

According to some embodiments of the invention, the thermally conducting layer is configured to provide a path along which heat from the heat source is conducted directly to an outer surface of the heat pack.

According to some embodiments of the invention, the thermally conducting layer is configured to facilitate rapid transfer of heat to an outer surface of the heat pack while the buffer is and/or remains unheated.

According to some embodiments of the invention, the thermally conducting layer is configured to facilitate rapid transfer of heat to an outer surface of the heat pack while sharing the heat with the buffer.

According to some embodiments of the invention, the buffer further includes a metallic matrix of high thermal conductivity.

According to some embodiments of the invention, the metallic matrix includes a net, screen, mesh, or metal foam.

According to some embodiments of the invention, the thermally conducting material includes small particles of a high thermally conducting material.

According to some embodiments of the invention, the thermally conducting material is located within the buffer, the heat source, surface stitch lines, or a combination thereof.

According to some embodiments of the invention, the thermally conducting material is located adjacent to the buffer, the heat source, surface stitch lines, or a combination thereof.

According to some embodiments of the invention, the stitch lines are configured to contact a body of a user.

According to some embodiments of the invention, the heat pack further includes an insulating layer.

According to some embodiments of the invention, the insulating layer at least partially encloses the heat source.

According to some embodiments of the invention, the insulating layer at least partially encloses the heat pack.

According to some embodiments of the invention, the heat pack further includes at least one baffle.

According to some embodiments of the invention, the at least one baffle is configured to contain the buffer or a portion thereof.

According to some embodiments of the invention, the heat pack further includes at least one pore configured for a passage of air to the heat source.

According to some embodiments of the invention, the heat pack further includes a temperature limiter controller configured to control a rate of reaction of the heat source.

According to some embodiments of the invention, the heat pack further includes a sensor configured to detect a temperature of the heat pack.

According to an aspect of some embodiments of the invention, there is provided a method for controlling a temperature of an outer surface of a heat pack, the method including: supplying heat with a heat source; absorbing the heat to a highly thermally conductive material; absorbing a first portion of the heat from the highly thermally conductive material to a buffer including a phase changing material configured to undergo an endothermic phase transition at a pre-defined temperature; and conducting a second portion of the heat along the highly thermally conductive material to an outer surface of the heat pack.

According to some embodiments of the invention, the first portion of the heat is greater than the second portion of the heat.

According to some embodiments of the invention, the first portion of the heat is similar to the second portion of the heat.

According to some embodiments of the invention, the first portion of the heat is less than the second portion of the heat.

According to some embodiments of the invention, the highly thermally conductive material provides a path along which heat from the heat source is conducted directly to a body of a user.

According to some embodiments of the invention, the highly thermally conductive material provides a path along which heat from the heat source is conducted directly to an outer surface of the heat pack.

According to some embodiments of the invention, the method further includes controlling a rate of an exothermic reaction of the heat source.

According to some embodiments of the invention, the controlling the rate of the exothermic reaction is by controlling a quantity of a regent available for reaction.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I: Schematic diagrams illustrating various exemplary heat packs, in accordance with some embodiments.

FIG. 2A: A chart of temperature v, time at Stage I and Stage II measured within the heat pack and an outer surface of the heat pack, in accordance with some embodiments.

FIG. 2B: A flow chart of heat flow at Stage I, in accordance with some embodiments.

FIG. 3: A flow chart of heat flow at Stage II, in accordance with some embodiments.

FIG. 4: A chart showing the buffer temperature verses heat in accordance with some embodiments.

FIG. 5: A chart showing the buffer temperature verses heat, in accordance with some embodiments.

FIG. 6A-C: Exemplary chemical reactions showing various methods for controlling an exothermic chemical reaction, in accordance with some embodiments.

FIG. 7: A schematic diagram illustrating an air flow controller to control the passage of air into the heat pack, in accordance with an embodiment.

FIG. 8: A schematic diagram illustrating an air flow controller to control the passage of air into the heat pack, in accordance with an embodiment.

FIG. 9A: A graph of heat versus temperature illustrating the buffer temperature verses heat, in accordance with some embodiments

FIG. 9B: A flow chart of heat flow illustrating the buffer temperature verses heat, in accordance with some embodiments

FIGS. 10A-C: Schematic diagrams illustrating a heat pack with reversible pore opening and closing, in accordance with some embodiments.

FIG. 11: A schematic diagram illustrating a heat pack, in accordance with some embodiments.

FIG. 12: A schematic diagram illustrating a cross-section view of a heat pack, in accordance with some embodiments.

FIG. 13: A schematic diagram illustrating a perspective view of a heat pack, in accordance with some embodiments.

FIG. 14: A block diagram illustrating a heat pack, in accordance with some embodiments.

FIG. 15: A flow chart of a method of using a heat pack, in accordance with some embodiments.

FIG. 16: A flow chart of a method of using a heat pack, in accordance with some embodiments.

FIG. 17: A flow chart illustrating a method of providing controlled heat, in accordance with some embodiments.

FIG. 18: A flow chart illustrating a method of providing controlled heat, in accordance with some embodiments.

FIGS. 19A-B: Schematic diagrams illustrating anatomic design of a heat pack and use thereof, in accordance with some embodiments.

FIGS. 20A-B: Schematic diagrams illustrating anatomic design of a heat pack and use thereof, in accordance with some embodiments.

FIG. 21: A block diagram illustrating a heat pack, in accordance with some embodiments.

FIG. 22: A flow chart for controlling the temperature of an outer surface of a heat pack, in accordance with some embodiments.

### DESCRIPTION OF SPECIFIC EMBODIMENTS OF THE INVENTION

The present invention in some embodiments thereof relates to a system and method for controlling the surface temperature of a heat pack.

### Overview

Some embodiments relate to an exothermic chemical heat pack which may be used to heat a user. Optionally, the heat pack may heat the skin of a user.

The current invention in some embodiments thereof, includes a rate limited chemical heat pack. According to some embodiments, the heat pack may be easily transportable, simple to use and/or flexible enough to conform to the shape of the body of the user.

Commonly, exothermic oxidation of iron is used as a heat source. A problem with such devices is that they can heat the skin to damaging temperatures (e.g., the maximum temperature may not be limited to less than a temperature ranging between 41° C. to 43° C.).

According to some embodiments, the rate of the chemical reaction may be controlled by limiting the supply of one or more reagents in the chemical reaction. For example, one or more pores in the heat pack may be opened and closed passively (e.g., using a thermocouple) and/or actively (e.g., using a controller). Alternatively, and/or additionally, a buffer may be used to absorb excess heat and/or release heat. Optionally, the buffer may be a phase changing material (PCM). For example, phase changing material may be used which changes phase at about 41° C. Optionally, the phase changing material may include paraffin (melting point of about 44° C.).

According to some embodiments, the system may include one or more heat conductive pathways. Optionally, one or more heat conductive pathways may be configured to transfer heat to a user's body rapidly. Optionally, the system may include one or more heat conductive pathways that may interlink and/or mix heat between the heat source and an outer surface of the heat pack. Optionally, the system may include one or more heat conductive pathways that may interlink and/or mix heat between the heat source and an outer surface of the heat pack and the buffer (phase changing material). Optionally the heat conductive pathway may include one or more thermally conducting materials. Optionally, the one or more thermally conducting materials may be highly thermally conducting, e.g., copper, silver, aluminum, graphene, diamond, boron nitride, carbon nanotubes, etc., and any combination thereof. Optionally, the thermally conducting material may have a thermal conductivity ranging between about 200 W/m K to about 500 W/m K, and/or between about 500 W/m K to about 1,000 W/m K, and/or between about 1,000 W/m K to about 3,000 W/m K, and/or between about 3,000 W/m K to about 7,500 W/m K.

According to some embodiments, the heat conductive pathway may be configured to facilitate rapid transfer of heat to an outer surface of the heat pack. Optionally, the heat conductive pathway may be configured to facilitate rapid transfer of heat to an outer surface of the heat pack even while the buffer is and/or remains cool. Optionally, the heat conductive pathway may be configured to facilitate rapid transfer of heat to an outer surface of the heat pack while sharing the heat with the buffer. Optionally, the heat conductive pathway may be a short circuit (e.g., stitch layer, etc.).

According to some embodiments, the heat pack may include one or more stitch lines or zones. Optionally, the stitch lines or zones may be heat conducting pathways. Optionally, the stitch lines may include a highly thermally conducting material. Optionally, stitch lines may directly connect between the heat source and the user's body. Optionally, the stitch lines may be configured to transfer heat rapidly to the user's body from a heat source.

According to some embodiments, the stitch lines may interlink and/or mix heat between the heat source and an outer surface of the heat pack. Optionally, the stitch lines may interlink and/or mix heat between the heat source and an outer surface of the heat pack and the buffer (phase changing material). Optionally, the stitch lines may be configured to facilitate rapid transfer of heat to an outer surface of the heat pack even while the buffer is and/or remains cool. Optionally, the stitch lines may be configured to facilitate rapid transfer of heat to an outer surface of the heat pack while sharing the heat with the buffer.

According to some embodiments, the stitch lines may be backed by a phase changing material. Optionally, the surface area of the stitch lines which may be in contact with the user's body may range between about 2% to about 40%, and/or between about 3% to about 25%, and/or between about 4% to about 12%.

According to some embodiments, the buffer may be configured to absorb heat e.g., while the heat source is producing heat. Optionally, the buffer may be configured to absorb heat from the heat source to prevent and/or reduce overheating of an outer surface of the heat pack. Optionally, the buffer may be configured to release absorbed heat as the heat source stops and/or slows heat production. Optionally, the buffer may be configured to release absorbed heat as the heat source stops and/or slows heat production thereby preserving the temperature of an outer surface of the heat pack for some time after the exothermic reaction has slowed and/or stopped.

In some embodiments, small particles of a high thermally conducting material may be added to the buffer e.g., phase changing material. Optionally, the high thermally conducting material may be a metal, e.g., copper, aluminum, iron, steel, zinc, bronze, nickel, lead, silver, graphene, carbon nanotubes, boron nitride, etc. Optionally, the particles may be added to the liquid phase changing material during production. Optionally, the particles may be added to the phase changing material to achieve a higher thermal conductivity of the solid PCM, and/or faster heat transfer to and/or from PCM. Optionally, addition of the particles may facilitate faster temperature rise and/or stable temperature during user heating. Optionally, the particles may range in size between about 0.01 μm to about 0.1 μm, and/or between about 0.1 μm to about 100 μm, and/or 100 μm to about 500 μm.

In some embodiments, the phase changing material may include a metallic matrix. Optionally, the metallic matrix may be a net and/or screen and/or mesh and/or metal foam. Optionally, the presence of the metallic matrix in the phase changing material may increase heat conductivity, e.g., disperse heat within the buffer. Optionally, the presence of a metallic matrix in the phase changing material may increase efficiency and/or work more rapidly. Optionally the matrix material may be soaked within the phase changing material during production.

According to some embodiments, the exothermic chemical reaction may be a lime-water reaction. Optionally, the exothermic chemical reaction may include reactive nanolaminates (such as, nickel-aluminum, titanium-boron, etc.). Optionally, the exothermic chemical reaction may include iron powder oxidation. According to some embodiments, the preferred exothermic chemical reaction may be iron powder oxidation. Optionally, iron powder oxidation may be controlled by limiting the amount of iron and/or oxygen available for reaction.

A first aspect according to some embodiments relates to a heat pack wherein an exothermic chemical reaction may be permitted to run uncontrolled and using a Phase Changing Material buffer between the user and the chemical reaction. According to some embodiments, the buffer may act as a heat sink, absorbing excess heat. For example, the PCM buffer may change phase from a first phase to a second phase in an endothermic phase transition. Optionally, the phase transition may be melting, crystallization, fusion, freezing, evaporation, sublimation, or any combination thereof.

According to some embodiments, the phase changing material buffer may be a material that changes phase from a first phase (e.g., gel) to a second phase (e.g., crystalized form) at a specific temperature (e.g., 41° C.). Optionally, the phase transition may be melting, crystallization, fusion, freezing, chemical reaction, etc. Optionally, the phase changing material may undergo a phase transition at a temperature ranging between about 38° C. to about 50° C. and/or between about 40° C. to about 45° C., and/or between about 41° C. to about 43° C.

According to some embodiments, when the chemical reaction releases sufficient heat to raise the temperature of the heat pack, and/or a portion thereof above a pre-defined temperature, the temperature of the phase changing material may be raised sufficiently to trigger a phase change of the phase changing material from a first phase to a second phase. Optionally, this phase transformation may be endothermic. Optionally, this phase transformation may be endothermic (e.g., melting, chemical reaction, alkane cracking, thermal decomposition, dissolution of a compound in an aqueous solution, etc.). Optionally, the phase changing material may protect the heat pack from overheating and/or damaging the skin of the user. Optionally, once the chemical reaction stops and/or the temperature of the heat pack decreases, the phase changing material may transform back to the first phase from the second stage. Optionally, transforming from the second phase to the first phase may release heat, e.g., in an exothermic reaction.

According to some embodiments, the phase changing material may absorb less heat per mass unit than is produced by the chemical reaction. According to some embodiments, a relatively large amount of phase changing material buffer may be needed relative to the amount of chemical fuel in the fuel source.

According to some embodiments, the weight ratio of phase changing material:chemical fuel may range between about 15:1 to about 3:1, and/or between about 5:1 to about 1:5, and/or between about 3:1 to about 1:3, and/or between about 5:1 to about 1:1.

According to some embodiments, the heat pack may remain hot for a several hours (e.g., 2-4). Optionally, such heat packs may be useful in a hospital setting, and/or for ambulances, and/or home use, and/or rescue use, and/or military use, etc.

According to some embodiments, phase changing material may be packaged in baffles (e.g., baffle stitched, baffle boxed, etc.). Optionally, packaging the phase changing material and/or fuel source may prevent and/or reduce accumulation of the heat source and/or buffer in one area of the heat pack, e.g., the packaging may prevent leaving an empty space which may result in contact of the hot chemical fuel with the skin, causing burns.

According to some embodiments, a single use heat pack may include an enclosed inner core of a chemical fuel surrounded by a phase changing material buffer with an outer chemical heating layer, which may optionally be attached to the skin of a user.

According to some embodiments, a single use heat pack may include a single use heat pack, wherein small chemical heating sections may be dispersed within a phase changing material buffer reservoir. Optionally, the heat pack may be attached to the skin of a user.

According to some embodiments, a reusable phase changing material buffer may connect to a single use chemical fuel heat pack.

According to some embodiments, the surface area of an outer surface of the heat pack which may be in contact with the user's body may range between 2% to about 90%, and/or between about 20% to about 75%, and/or between about 30% to about 60%.

According to a second aspect of some embodiments, the temperature of the heat pack may be controlled by controlling the rate of an oxidation reaction (e.g., oxygenation of iron). Optionally, the rate of oxidation may be limited by limiting the oxygen supply to the heat pack. For example, when the temperature of the heat pack rises above a pre-determined temperature, the oxygen supply may be limited thereby reducing the reaction rate.

According to some embodiments, the heat pack may be encapsulated by a porous layer. Optionally, the porous layer may include one or more pores on one or more surfaces, e.g., an upper surface. Optionally, the porous layer may expand on heating, thereby sealing the one or more of the pores. Optionally, the pores may include valves which may be operated manually and/or automatically. Optionally, the user may place a cover over one or more pores when the heat pack reaches a pre-determined temperature, e.g., preventing the heat pack from getting too hot. Optionally, the one or more pores may be covered manually and/or automatically once a pre-determined temperature is reached.

According to some embodiments, sealing one or more pores may reduce and/or prevent oxygen from entering the heat pack. Optionally, sealing one or more pores may reduce the rate of and/or stop the exothermic chemical reaction. Optionally, sealing one or more pores may reduce the temperature of the heat pack. Optionally, if the heat pack becomes too cold, the one or more pores may be unsealed to increase the amount of oxygen entering the heat pack, and/or increase the rate of reaction, and/or increase the amount of heat thereby produced by the exothermic chemical reaction.

According to some embodiments, the heat pack may include an adhesive layer. For example, the adhesive layer may attach the heat pack to the user's body. Optionally, the skin of the user may be adhered directly to a high heat conduction layer of the heat pack. Optionally, this may facilitate heat transfer through conduction.

In contrast, a heat pack that may heat to temperatures that can cause burns is often separated from the body, transferring the heat only an insulating layer and/or via convection. Advantageously, by direct contact, some embodiments of the current invention may transfer greater amounts of heat, faster, using conduction than traditional insulated heat packs.

Some embodiments of the current invention may be configured for heat to be transferred through convection from the external layer (not close to the body). For example, heat may be supplied to the space around the user's body, by various means, including through conduction.

According to some embodiments, the surface area of the heat pack which may be in contact with the user's body may range between 2% to about 90%, and/or between about 20% to about 75%, and/or between about 30% to about 60%. Optionally, the heat pack may include one or more stitch zones. Optionally, the stitch zones may include a high conductivity material. Optionally, stitch zones may directly connect between the heat source and the user's body. Optionally, the stitch zones may transfer heat rapidly to the user's body. Optionally, the stitch zones may be backed by a chase changing material. Optionally, the surface area of the stitch zones which may be in contact with the user's body may range between about 2% to about 40%, and/or between about 3% to about 25%, and/or between about 4% to about 12%.

In some embodiments, the heat pack may include various layers. Optionally, the heat pack may include deep and/or shallow layers relative to the surface of the heat pack. Optionally, the heat pack may include heating and/or buffering layers.

In some embodiments, the heat pack may include a highly conductive layer. Optionally, the highly conductive layer may facilitate maintaining a constant temperature over space and/or time. Optionally, the highly conductive layer may facilitate achieving a temperature that is averaged between different portions of the heat pack. Optionally, the highly conductive layer may facilitate transfer of heat rapidly between the heat pack to the user's skin and/or from the user's skin to a heat buffer. For example, a high conductivity layer may connect a skin contact surface to deep layers of the heat pack and/or connect between a skin contact area and different layers of the heat pack simultaneously (e.g., both deep and shallow layers and/or both heated and buffered layers). Optionally, adhesive may be applied to a highly conductive layer.

According to some embodiments, the high thermal conductivity layer may be covered, at least in part by a phase changing material layer. Optionally, the surface area of the heat pack in which the high thermal conductivity layer may not be covered by the phase changing material may range between about 2% to about 40%, and/or between about 3% to about 25%, and/or between about 4% to about 12%.

According to some embodiments, the heat pack may include one or more sensors. Optionally, the sensors may include heat sensors, e.g., thermocouples, etc. Optionally, the one or more sensors may be connected to a controller. Optionally, the controller may open and/or close one or more pores on a surface of and/or within the heat pack in response to a temperature detected by one or more sensors. Optionally, closing one or more surface pores may reduce and/or prevent oxygen from entering the heat pack. Optionally, closing one or more pores may reduce the rate of the exothermic chemical reaction. Optionally, closing one or more pores may stop the exothermic chemical reaction. Optionally, closing one or more pores may reduce the temperature of the heat pack.

According to a third aspect of some embodiments, the temperature of the heat pack may be controlled by controlling the rate of the chemical reaction by controlling the supply of fuel (e.g., the iron). Optionally, the fuel may be the heat source for the exothermic reaction.

According to some embodiments, the heat pack may include one or more sensors. Optionally, the sensors may include heat sensors, e.g., thermocouples, etc. Optionally, the one or more sensors may be connected to an integrated controller (e.g., an integrated circuit and/or electronic controller). For example, an electronic controller may respond to a temperature sensor. Optionally, when the heat pack is too cool it may release material to fuel the chemical reaction. For example, if the exothermic chemical reaction is iron oxidation the controller may release portions of iron to be oxidized.

According to some embodiments, the weight ratio of phase changing material:chemical fuel may range between about 5:1 to about 1:5, and/or between about 3:1 to about 1:3, and/or between about 5:1 to about 1:1.

According to some embodiments, such heat packs may remain hot for between 1 to 2 hours and/or between 2 to 4 hours and/or between 4 to 8 hours and/or between 8 to 24 hours. Optionally, the heat packs may be used for military and/or mountain rescue operations, e.g., where the heat pack may need to be very easy to transport and where a user may need significant heating over a long time before they can be transported to safety.

According to some embodiments, a buffer and/or fuel source may be packaged in baffles (e.g., baffle stitched, baffle boxed, etc., e.g., like a duvet). Optionally, packaging the phase changing material and/or fuel source may not gather in one area or the heat pack leaving an empty space which may result in contact of the hot chemical fuel with the skin, causing burns.

According to some embodiments, the heat pack may include a combination of any of the previous aspects described herein.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

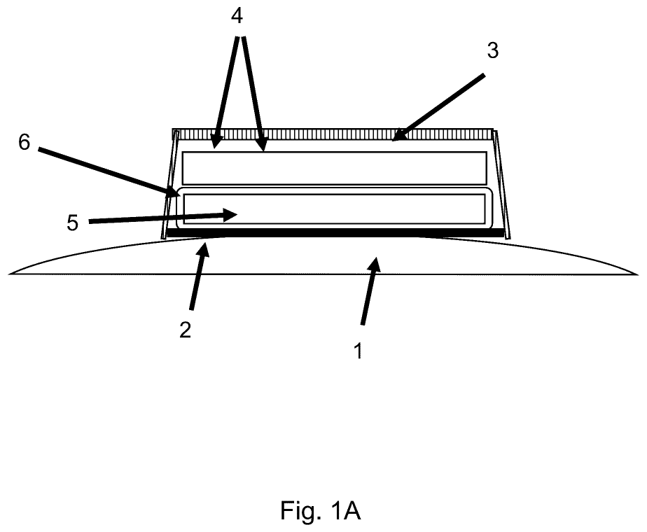

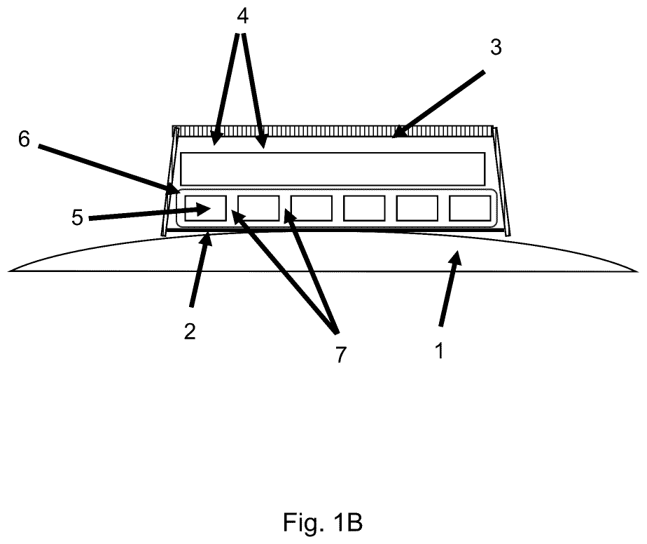

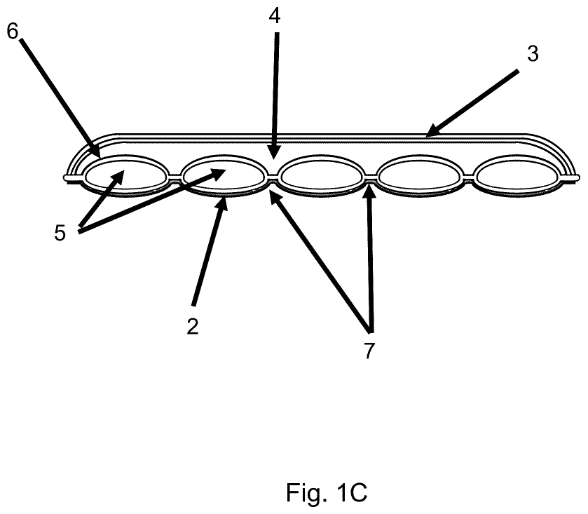

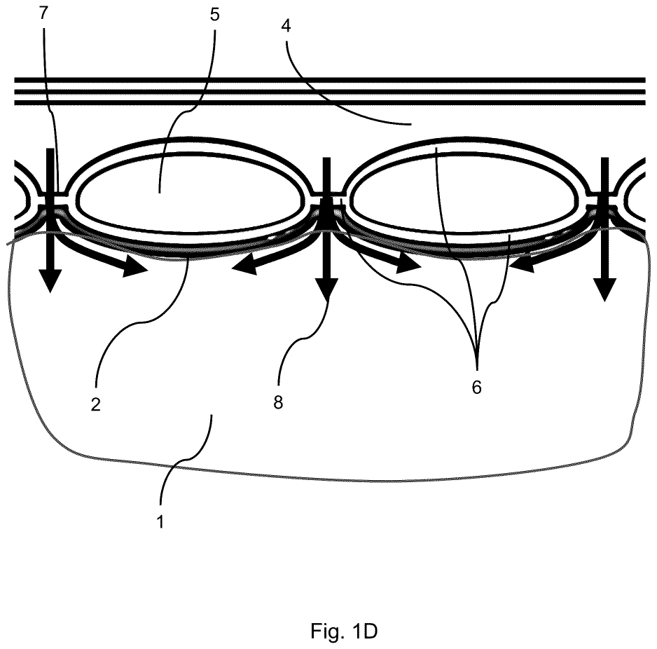

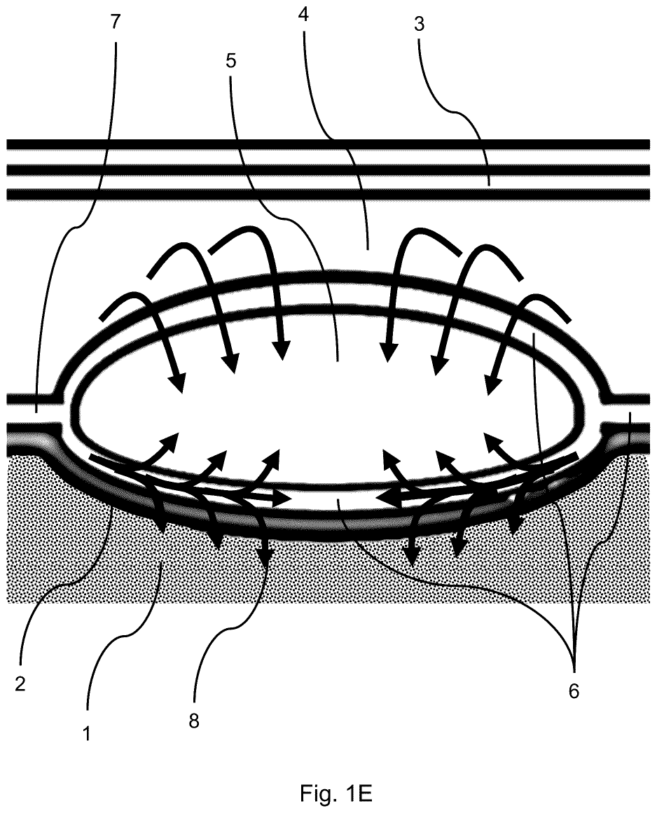

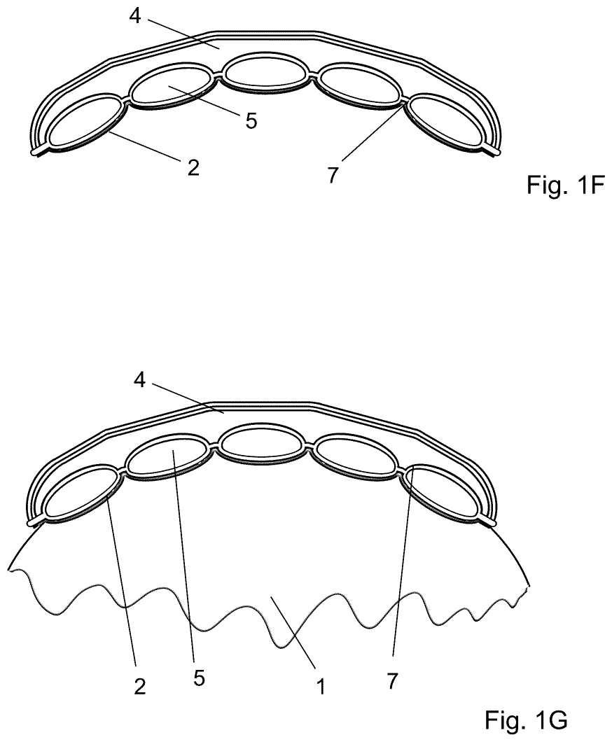

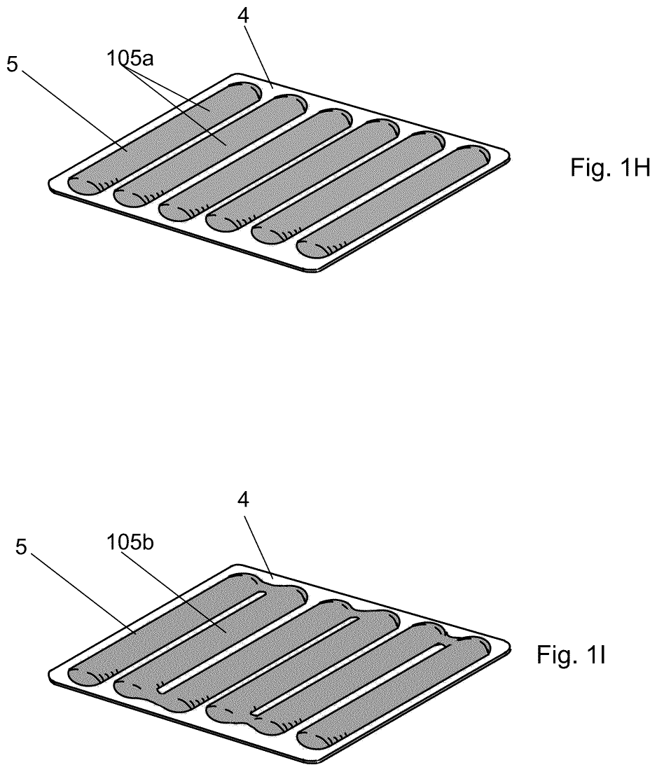

FIG. 1A is a schematic diagram of a heat pack, in accordance with some embodiments. For example, the heat pack may be placed and/or adhered to the skin of the body 1 of a user. For example, the skin contact region may include a high thermal conductivity layer and/or an adhesive 2. The heat pack may include an isolation barrier 3. For example, the isolation barrier 3, may be impermeable to heat and/or water. Optionally, the isolation barrier 3 may be permeable to oxygen. The isolation barrier 3 may enclose an exothermic mixture 4 (e.g., a fuel source). Optionally, a buffer 5 may separate between the exothermic mixture 4 and the skin contact surface. For example, the buffer 5 may include a material that absorbs and/or stores excess heat when the exothermic mixture gets to hot (e.g., over about 43 degrees C.) and/or preserves heat at a desired temperature (e.g., releasing heat when the body contact surface goes below a desired temperature (e.g., less than about 43 degrees C.). For example, the buffer 5 may include a phase change material that absorbs heat and changes phase (e.g., melts) when heated past a phase change temperature (e.g., between 41 to 44 degrees C.) and/or releases heat and/or changes phase (e.g., solidifies) when cooled past the phase change temperature. The buffer material 5 is optionally enclosed within and/or contacts a high thermal conductivity material 6. Optionally, heat transfer 8 to the body 1 of the user may be convection and/or conduction. For example, buffer material and/or fuel source may be packaged in baffles (e.g., baffle stitched, baffle boxed, etc., e.g., like a duvet). Optionally, packaging the phase changing material and/or fuel source may not gather in one area or the heat pack leaving an empty space which may result in contact of the hot chemical fuel with the skin, causing burns.

FIG. 1B is a schematic diagram of a heat pack with conduction lines passing through a buffer layer, in accordance with some embodiments. For example, the heat pack may be placed and/or adhered to the body 1 (e.g., the skin) of a user, e.g., by a high thermal conductivity layer, which may include adhesive 2. The heat pack may include isolation barrier 3 which may be impermeable to heat and/or water. Optionally, isolation barrier 3 may be permeable to oxygen. Isolation barrier 3 may enclose an exothermic mixture 4 (fuel source) and/or buffer 5 comprising a phase change material. The buffer material 5 may be enclosed within and/or contact a high thermal conductivity material 6. Optionally, thermal conduction lines may include stitch-lines 7 between baffles of a heat absorbing buffer (e.g., PCM). Optionally, heat transfer 8 to the body 1 of the user may be convection and/or conduction. Optionally, stitch-lines 7 may include high thermal conductivity materials and/or layers. Optionally, stitch lines 7 may act to short circuit the buffer under some conditions. For example, without the high conduction lines, the adhesive may not heat up until the PCM buffer heats up, which may take a long time after the exothermic mixture has begun to produce heat. With the high conduction lines, when the exothermic mixture begins to heat up, the high conduction lines may facilitate quick heat transfer from the exothermic reaction to the high conduction and/or adhesive region and/or to the body. For example, while the buffer remains cool, heat is conducted along the high conductivity lines from the exothermic mixture to the high conductivity lines at the skin interface.

FIG. 1C illustrates an embodiment of a heat pack with stitching lines through the buffer layer, in accordance with an embodiment of the current invention. For example, the heat pack may be placed and/or adhered to the body 1 of a user, e.g., by a high thermal conductivity layer, which may include an adhesive 2. The heat pack may include isolation barrier 3 which may be impermeable to heat and/or water. Optionally, isolation barrier 3 may be permeable to oxygen. Isolation barrier 3 may enclose an exothermic mixture 4 (fuel source) and/or a buffer 5 comprising a phase change material. The buffer material 5 may be enclosed within a high thermal conductivity material 6. Optionally, stitch lines 7 may be stitch-lines between baffles. Optionally, the baffles may include a heat absorbing buffer (e.g., PCM). Optionally, heat transfer to the body of the user may be via convection and/or conduction. Stitch lines 7 may serve multiple functions. For example, the stitching lines may prevent the buffer material for bunching (e.g., like quilting line prevent batting of a quilt from bunching). For example, stitching may prevent buffer bunching away from large sections of the contact zone and/or allowing the exothermic mixture to come into contact with the contact zone and/or causing a burn to the subject. Optionally, along the stitching lines, heat from the exothermic mixture may short circuit the buffer areas and heat the body rapidly from the heat source, optionally prior to heating the buffer.

FIG. 1D illustrates heat transfer in an embodiment of a heat pack with stitching lines through the buffer layer, in accordance with an embodiment of the current invention. For example, the heat pack may be placed onto and/or adhered to the body 1 of a user, e.g., by a high thermal conductivity layer, which may include adhesive 2. The heat pack may include isolation barrier 3 which may be impermeable to heat and/or water. Optionally, isolation barrier 3 may be permeable to oxygen. Isolation barrier 3 may enclose an exothermic mixture 4 (fuel source) and/or buffer 5. Optionally, the buffer 5 comprises a phase change material. Buffer 5 may be enclosed within and/or contact a high thermal conductivity material 6. Optionally, thermal conduction lines may be stitch lines 7 between baffles. Optionally, the baffles may include a heat absorbing buffer (e.g., PCM). Optionally, heat transfer 8 to the body 1 of the user may be convection and/or conduction. There may be multiple mechanisms affecting the transfer of heat quickly from the exothermic mixture. For example, the heat not only moves to the body through the PCM, but also transfers through the stitches between the PCM filled areas, this allows the heat transfer to the body short circuiting the PCM. Thus, the body may start to warm while the PCM is still equilibrating and/or before the PCM heats to an optimal temperature.

FIG. 1E illustrated heat transfer between various structures in a heat pack, in accordance with an embodiment of the current invention. For example, the heat pack may be placed and/or adhered to the body 1 of a user, e.g., by a high thermal conductivity layer, which may include an adhesive 2. The heat pack may include an isolation barrier 3 which may be impermeable to heat and/or water. Optionally, the isolation barrier 3 may be permeable to oxygen. The isolation barrier 3 may enclose an exothermic mixture 4 (fuel source) and/or a buffer 5 comprising a phase change material. The buffer material 5 may be enclosed within a high thermal conductivity material 6. Optionally, thermal conduction lines may be stitch-lines 7 between baffles of a heat absorbing buffer (e.g., PCM). Optionally, heat transfer 8 to the body 1 of the user may be convection and/or conduction. The highly conducting material 6 may serve as a short circuit allowing heat to reach the body without passing through PCM buffer 5. Additionally, or alternatively, the highly conducting material 6 may act as a heat sink that equalizes heating from the exothermic mixture 4 and cooling by the buffer 5. Optionally, the highly conductive material 6 may have a large surface in contact with the exothermic material and/or a large surface in contact with the PCM buffer and a large surface area in contact with the body. Optionally, by adjusting the sizes of these contact surfaces and/or the size of the short-circuiting stitch lines, the spread of heat may be balanced. For example, heat is spread and drained to the PCM facilitating quick heating of the body while inhibiting overheating of areas in contact with the skin and/or causing burns.

FIGS. 1F and 1G illustrate a heat pack, in accordance with an embodiment of the current invention. For example, the stitch line structures 7 may facilitate flexibility of the skin contact surface. Optionally, the stitch line structures 7 may facilitate flexibility of the skin contact surface when the PCM is cold and/or resistant to bending. Optionally, the stitch line structures 7 may facilitate flexibility of the skin contact surface when the PCM is warm and/or resistant to bending.

FIGS. 1H and 1I illustrate various exemplary buffer configurations in a heat pack, in accordance with an embodiment of the current invention. For example, the stitch lines may isolate baffles 105a of the buffer (e.g., as illustrated in FIG. 1H). Alternatively or additionally, the buffer in multiple baffles 105b may be interconnected (e.g., as illustrated in FIG. 1I). Optionally, the buffer may be contained within a single baffle. Optionally, the baffles may have various configurations, e.g., zigzagging lines, coil, stripes, interlocking “U”s, etc. Optionally, the baffles and/or stitch lines may facilitate heat transfer. Optionally, the baffles and/or stitch lines may facilitate flexibility of the skin contact surface of the heat pack.

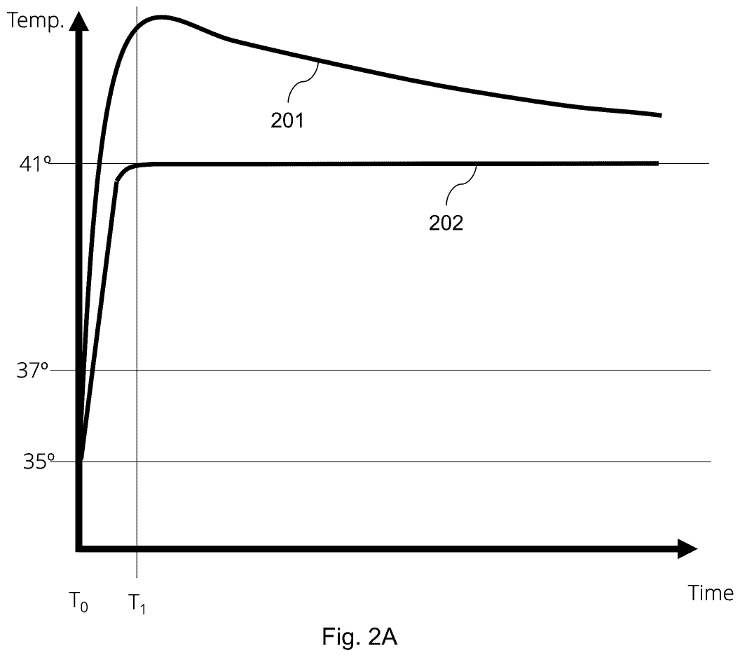

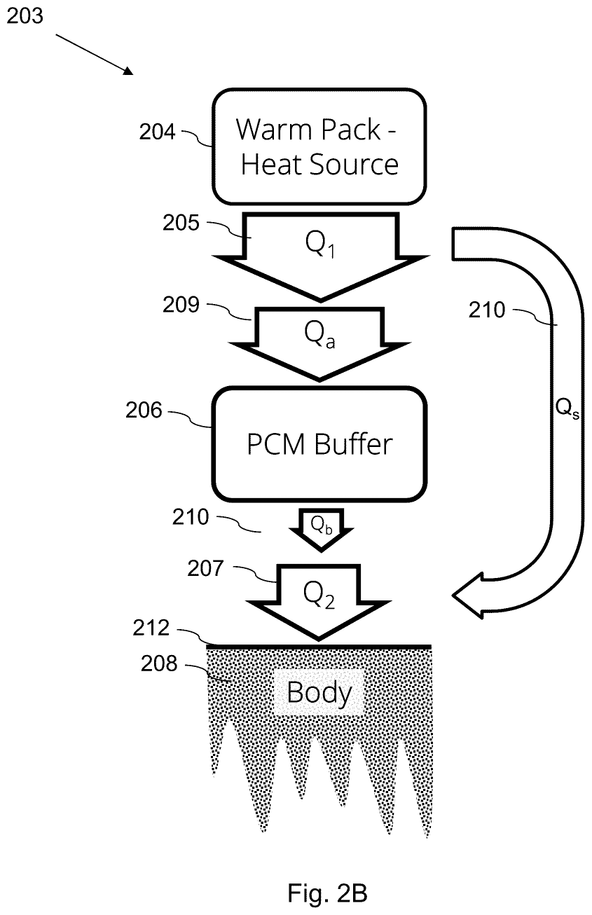

FIG. 2A is a graph of temperature v, time measured withing the heat pack (graph 202) and an outer surface of the heat pack (graph 202), in accordance with some embodiments. For example, the graph illustrates stage 1, the acceleration from initiation (at T₀) of the exothermic chemical reaction until a desired temperature is reached at T₁. After initiation of the exothermic chemical reaction, the exothermic chemical reaction may accelerate to a peak temperature and then slowly reduce. The temperature within the exothermic reactants (as shown by graph 201) is significantly higher and more variable than the temperature on an outer surface of the heat pack (as shown by graph 202).

FIG. 2B is a flow chart of heat flow at Stage I, in accordance with some embodiments. For example, in method 203, the heat pack is heated by heat transferred (Q₁) 205 from the heat source 204. The subject's 208 body is heated by heat transfer (Q₂) 207 from the outer surface 212 of the heat pack. Optionally, at this stage the heat transfer (Qₐ) 209 (absorbed) to the buffer 206 may be larger than the heat transferred (Q_{b}) 210 from the buffer 206 through the outer surface 212 to the subject 208, i.e., the heat source 204 may have a heat transfer Q₁ 205 which is larger than the heat transfer Q₂ 207 to the subject 208. For example, because during this stage, the phase changing material buffer 206 may absorb more heat Qₐ from the heat source than the heat transferred Q_{b} by the buffer 206 to the user 208, i.e., Q₁>Q₂. Optionally, at this stage, the buffer 206 is getting warmed.

Optionally, the heat (Q₁) 205 transferred from the heat source 204 includes heat (Qₐ) 209 absorbed by the buffer 206 and heat (Qₛ) 210 short circuited (e.g., transferred through the high conductivity path) to subject 208 through the outer contact surface 212 of the heat pack (Q₁=Qₛ+Qₐ). Optionally, the heat (Q₂) 207 transferred across the contact surface 212 to the subject 208 may include heat (Qₛ) 210 passing from the heat source along the high conductivity path short circuiting the PCM buffer 206 and heat (Q_{b}) 210 transferred from the PCM buffer to outer surface 212 and the subject 208. (i.e., Q₂=Q_{b}+Qₛ). Optionally, at this stage the buffer 206 is absorbing more heat than it is transferring to the subject Qₐ>Q_{b} and/or the heat transferred to the subject is less than that heat produced by the heat source Q₂<Q₁). For example, as heat Qₛ is transferred from the exothermic reaction along high conductivity paths, heat is absorbed by the cool buffer 206 (which is optionally also in contact with the high conductivity path). Optionally, the temperature of the outer surface 212 of the heat pack in contact with the subject 208 may higher than the temperature of the buffer 206 and/or lower than the temperature of the exothermic reactants inside the heat pack. for example, while the temperature within the heat source 204 is above about 41° C., the surface temperature of the heat pack on the skin of the subject 208 is about 41° C. In some embodiments, short circuiting heat Qₛ may facilitate the heat pack quickly starting to supply significant heat to the subject while the buffer is stilling warming.

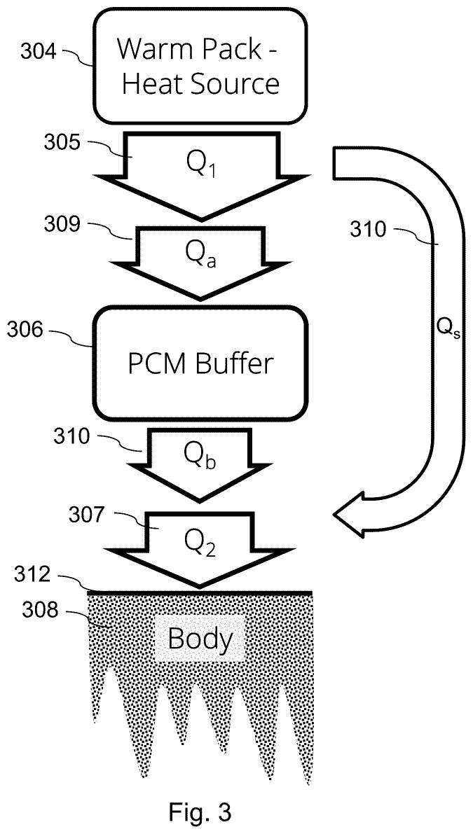

FIG. 3 is a flow chart of heat flow at Stage II, in accordance with some embodiments. Temperature versus time at Stage II measured withing the heat pack and an outer surface of the heat pack, is illustrated in FIG. 2A in accordance with some embodiments. For example, at Stage II around the set point, the graph illustrates the acceleration from initiation (at T₀) of the exothermic chemical reaction until a desired temperature is reached at T₁. After initiation of the exothermic chemical reaction, the exothermic chemical reaction may accelerate to a peak temperature and then slowly reduce. The temperature (as shown by graph 201) within the heat source 304 is significantly higher and more variable than the temperature on an outer surface 312 of the heat pack (as shown by graph 202). However, the heat (Q₂) transferred across the surface 312 of the heat pack to the subject 308 may remain relatively steady. Additionally or alternatively the heat Qₐ 309 absorbed by the buffer may remain about the same and/or more than the heat Q_{b} 310 transferred from the buffer through the surface 312 to the subject 308, i.e., Q₁>˜Q₂, for example, while the temperature within the heat source 304 is above about 41° C., the surface temperature of the heat pack on the skin of the subject 308 and/or of the buffer 304 remains relatively constant at about 41° C. Optionally, the temperature of the surface 312 will be slightly greater than the temperature of the buffer 306 (e.g., due to heat Qₛ 310 short circuiting the PCM buffer 306 through the high conductivity path from the heat source 304 to the outer surface 312 and/or the subject 308.

In some embodiments, at Stage II around the set point, the heat pack is heated 304 by heat transfer (Q₁) 305 from the heat source to the buffer 306. The user's body 308 is heated by heat transfer (Q₂) 307 through the surface 312 of the heat pack. Optionally, heat transfer to the buffer Qₐ 308 may be greater than the heat Q_{b} transfer from the buffer 306 to the subject 308, i.e., the phase changing material of the buffer 306 may absorb heat and/or change phase while remaining at a steady temperature. Additionally or alternatively, as the temperature of the heat source 304 is reduced the heat Q₁ 305 transfer decreases as does heat Qₐ 309 transferred to the buffer and heat Qₛ 309 transferred along the high conductivity pathway directly to the surface 312 and subject 308. For example, a balance may be reached wherein the heat Qₐ 309 absorbed by the buffer 306 is approximately equal to the heat Q_{b} 310 transferred from the buffer through the surface 312 to the subject 308 and/or a balance may be reached wherein the heat Q₁ 305 transferred out from the heat source 304 is approximately equal to the total heat Q₂ 307 transferred from through the surface 312 to the subject 308. In a further stage, the fuel of the heat source 304 may run down and/or run out and/or the temperature of the heat source 304 may be reduced. As the temperature of the heat source 304 and/or the contact surface 312 is reduced below the desired temperature, the buffer 306 may continue to emit heat Q_{b} 310. For example, the heat Q_{b} emitted by the buffer 306 by be greater than the heat Qₐ 309 it receives from the heat source. Optionally, the temperature of the buffer 306 may be preserved e.g., by reversing the phase change (e.g., to melting) to release heat stored in the buffer. Optionally, the heat Q_{b} 310 released by the buffer may preserve the temperature of the contact surface 312 at a desired temperature (e.g., around 41 degrees C. (e.g., ranging between 40 to 43 degrees).

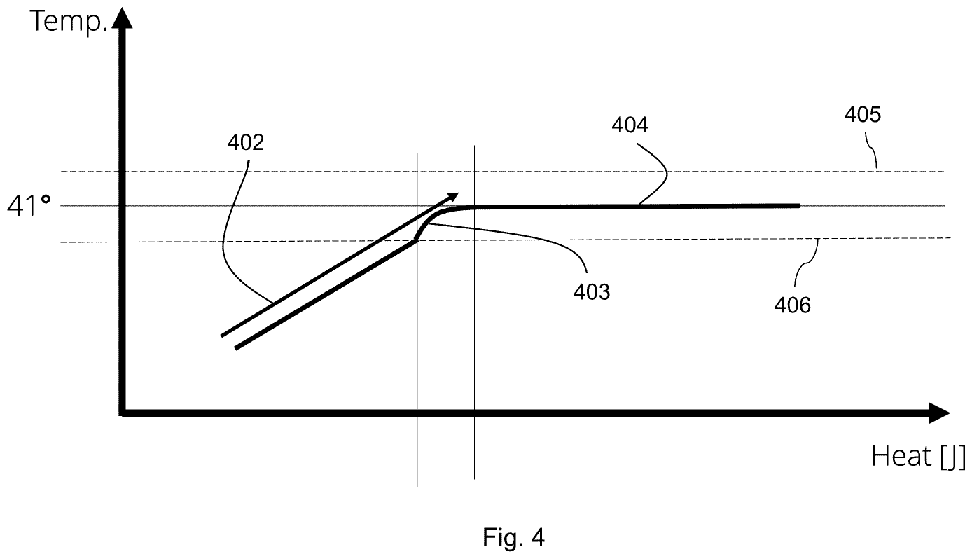

FIG. 4 is a chart showing the buffer temperature versus heat, in accordance with some embodiments. For example, in Step 1, the temperature of the buffer may increase during the acceleration phase 402 of the exothermic chemical reaction as it absorbs heat from the exothermic chemical reaction. In Step 2, the phase changing material may undergo a phase transition 403 from a first phase to a second phase (e.g., crystallization, solidification), once a specific temperature has been reached (e.g., approximately 41° C. (e.g., in a range between 40 to 44 degrees C.)). In Step 3, equilibrium 404 of the phase changing material may be reached as it repeatedly transforms from a first phase to a second phase and back to a first phase as the temperature fluctuates between a first temperature 405 and a second temperature 406, as heat is absorbed from the exothermic chemical reaction of the heat source, to heat and cool the heat pack, thereby maintaining the heat pack surface within a desired temperature range.

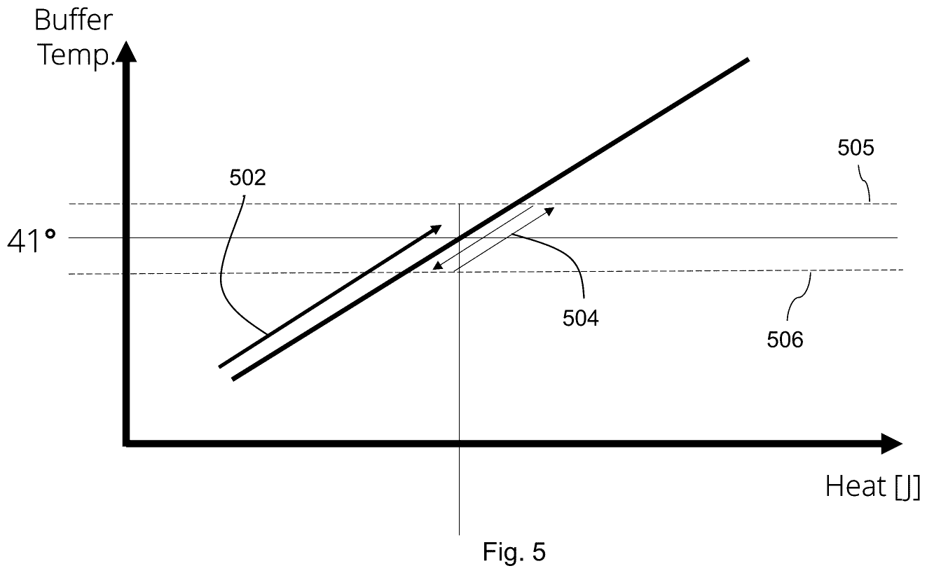

FIG. 5 is a chart showing the temperature verses heat, in accordance with some embodiments. For example, in Step 1, the temperature of the heat pack may increase during the acceleration phase 502 of the exothermic chemical reaction as it absorbs heat from the exothermic chemical reaction. In Step 2, a negative feedback loop 504 may slow the reaction in a deceleration phase. As the reaction slows, the temperature may be reduced and the feedback loop may allow the reaction rate to increase. This raises the temperature. The system may cycle repeatedly as it oscillates between the acceleration phase and the deceleration phase and back and/or the temperature fluctuates between a first temperature 505 and a second temperature 506. as heat is absorbed from the exothermic chemical reaction, to heat the pack and to allow it to cool, thereby maintaining the heat pack at a desired temperature (e.g., approximately 41° C. (e.g., in a range between 40 to 44 degrees C.)).

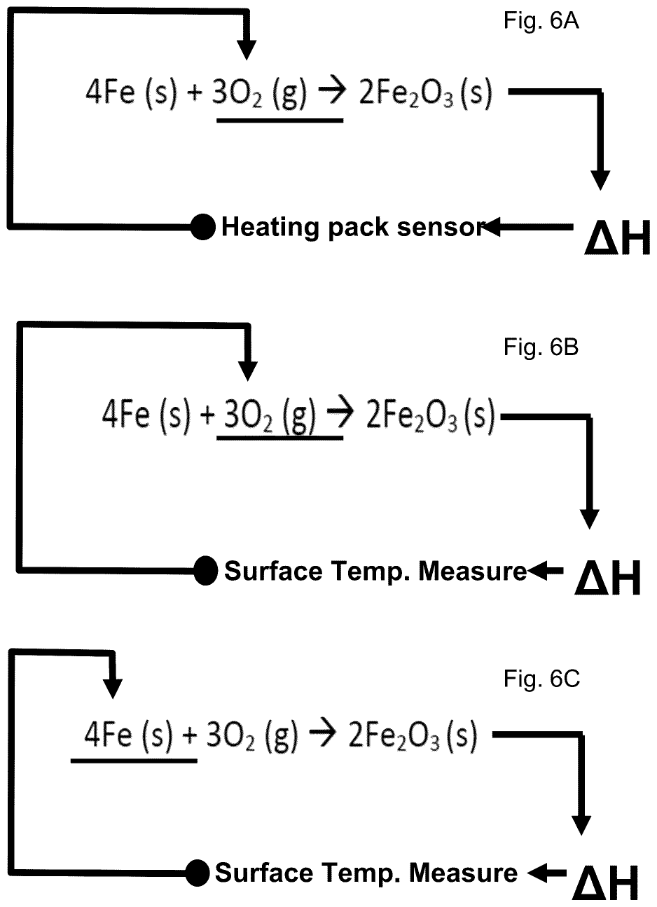

FIG. 6A-C are exemplary chemical reactions showing various methods for controlling an exothermic chemical reaction, in accordance with some embodiments. For example, the exothermic chemical reaction may be oxidation of iron.

FIG. 6A, illustrates and exemplary exothermic chemical reaction controlled by controlling the amount of oxygen available for reaction in accordance with an embodiment of the current invention. Optionally, the heat pack may include a temperature sensor within the bag. For example, when the detected temperature reaches a predetermined temperature, one or more pores in the heat pack may be closed to reduce the amount of oxygen available for reaction, thereby reducing the rate of the reaction and the amount of heat produced by the exothermic reaction.

FIG. 6B, illustrates and exemplary chemical reaction controlled by controlling the amount of oxygen available for reaction, in accordance with an embodiment of the current invention. For example, the surface of the heat pack may include a temperature sensor. Optionally, when the detected surface temperature reaches a predetermined temperature, one or more pores in the heat pack may be closed to reduce the amount of oxygen available for reaction, thereby reducing the rate of the reaction and the amount of heat produced by the exothermic reaction.

FIG. 6C, illustrates and exemplary chemical reaction controlled by controlling the amount of iron available for reaction. For example, the heat pack may include a temperature sensor. Optionally, when the detected temperature reaches a predetermined temperature, the amount of iron available in the heat pack may be reduced (e.g., by closing off one or more fuel sources, manually and/or automatically), thereby reducing the rate of the reaction and the amount of heat produced by the exothermic reaction.

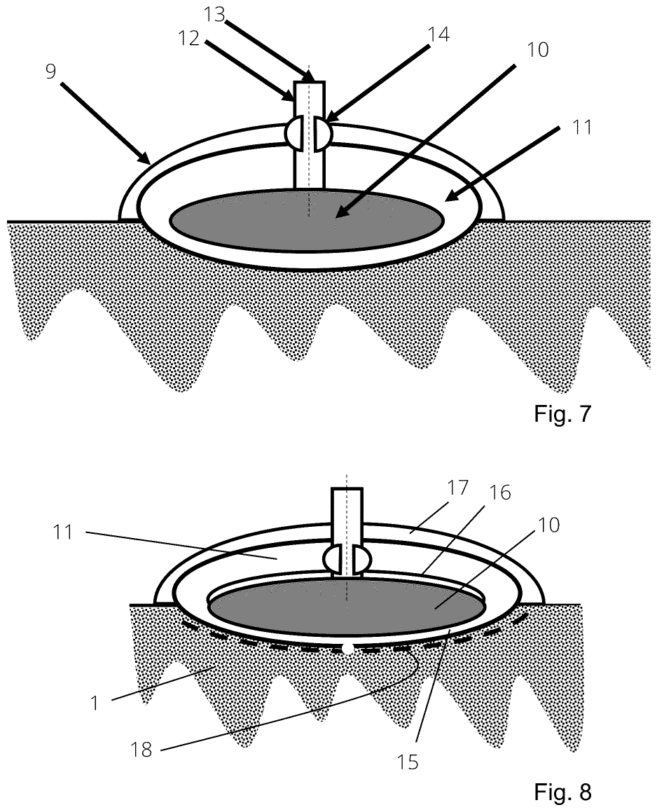

FIG. 7 is a schematic diagram showing an air flow controller to control the passage of air into the heat pack, in accordance with an embodiment. For example, the heat pack may include an isolation barrier with one or more pores. Optionally, each pore may include a passageway 12 linking the heat pack heat source 10 (e.g., chemical fuel) to the exterior of the heat pack. Optionally, the passageway 12 may pass through a buffer 11. Optionally, the passageway 12 may be impermeable to the buffer 11. Optionally, each pore may include an air entrance 13 and an air flow control 14, e.g., a valve. For example, air may pass through air flow control 14 from the air entrance 13 to the heat source 10 where it may be used in an exothermic reaction. Optionally, the air flow control 14 may be located within an isolation barrier 9 and/or within the buffer reservoir 11. For example, phase changing material and/or fuel source may be packaged in baffles (e.g., baffle stitched, baffle boxed, etc., e.g., like a duvet). Optionally, packaging the phase changing material and/or fuel source may not gather in one area or the heat pack leaving an empty space which may result in contact of the hot chemical fuel with the skin, causing burns.

FIG. 8 is a schematic diagram showing an air flow controller to control the passage of air into the heat pack, in accordance with an embodiment. For example, the heat source 10 may be symmetrically or asymmetrically located within the heat pack. When the heat source 10 is asymmetrically located within the heat pack, it may be located further and/or closer to the skin of the user. Optionally, when the heat source 10 is asymmetrically located within the heat pack, it may be separated from the user's body 1 by a thin buffer layer 15. Optionally, the thin buffer layer 15 may be fluidically connected to a thick buffer 11 which may serve as a heat sink or reservoir. Optionally, heat source 10 may be covered at least in part by an insulating and/or isolating barrier 16, 17. Optionally, the heat pack may be covered at least in part by an insulating and/or isolating barrier 17. Optionally, the heat pack may include a high surface area 18 in contact with the body 1 of the user.

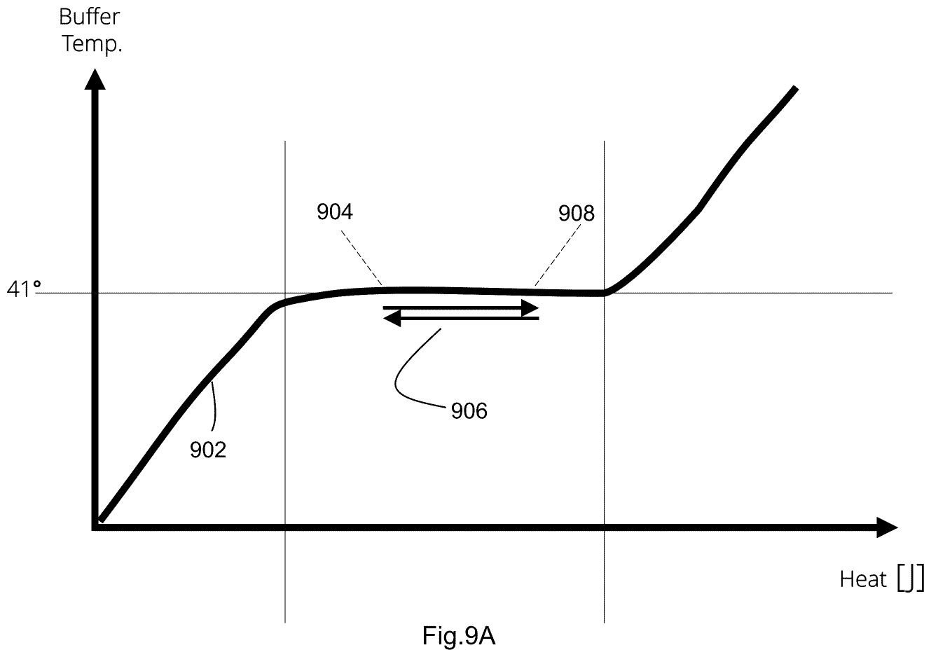

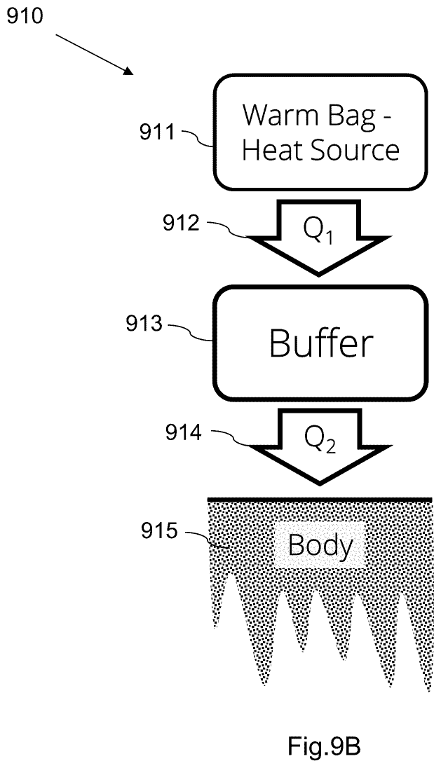

FIG. 9A is a graph of temperature versus heat of the Phase Changing Material, in accordance with some embodiments. Optionally, a heat pack may include an exothermic reaction, a negative feedback loop to control the reaction rate and a buffer (e.g., a PCM buffer. For example, the buffer may maintain a more even temperature at the skin contact surface than in the exothermic reaction itself. For example, in Step 1, 902 the temperature of the buffer may increase during the acceleration phase of the exothermic chemical reaction as it absorbs heat from the exothermic chemical reaction. In Step 2, a negative feedback loop 906 of the exothermic reaction may be reached as it repeatedly accelerates and decelerates. Nevertheless, the buffer maintains a relatively steady temperature as it absorbs heat when the heat source is hot and transforms from a first phase to a second phase and releases heat when the heat source is cool, transforming back to a first phase. In some embodiments, this maintains the heat pack at a desired temperature. When the detected temperature reaches and/or exceeds a predetermined temperature, one or more pores in the heat pack may be closed 904 to reduce the amount of oxygen available for reaction, thereby reducing the rate of the reaction and the amount of heat produced by the exothermic reaction. When the detected temperature reaches and/or falls below a predetermined temperature, one or more pores in the heat pack may be opened 908 to increase the amount of oxygen available for reaction, thereby increasing the rate of the reaction and the amount of heat produced by the exothermic reaction.

FIG. 9B is a flow chart of heat flow, in accordance with some embodiments. For example, in method 910, the heat pack is heated 911 by heat transfer (Q₁) 912 from the heat source to the buffer 913. The user's body 915 is heated by heat transfer (Q₂) 914 from the buffer 913. Optionally, heat transfer to the buffer may be similar to the heat transfer from the buffer to the user, i.e., the heat source may have a Q₁ which is similar to the Q₂ of a phase changing material buffer, which may absorb heat from the heat source, i.e., Q₁˜Q₂.

Optionally, the maximum heat capacity Q₁-Q₂ may be less than the phase changing material heat capacity, therefore, there may be no need for a valve and/or other means to open and/or close one or more pores. Optionally, Q₁-Q₂ may be greater than the phase changing material heat capacity, therefore, there may be a need for a valve and/or other means to open and/or close one or more pores.

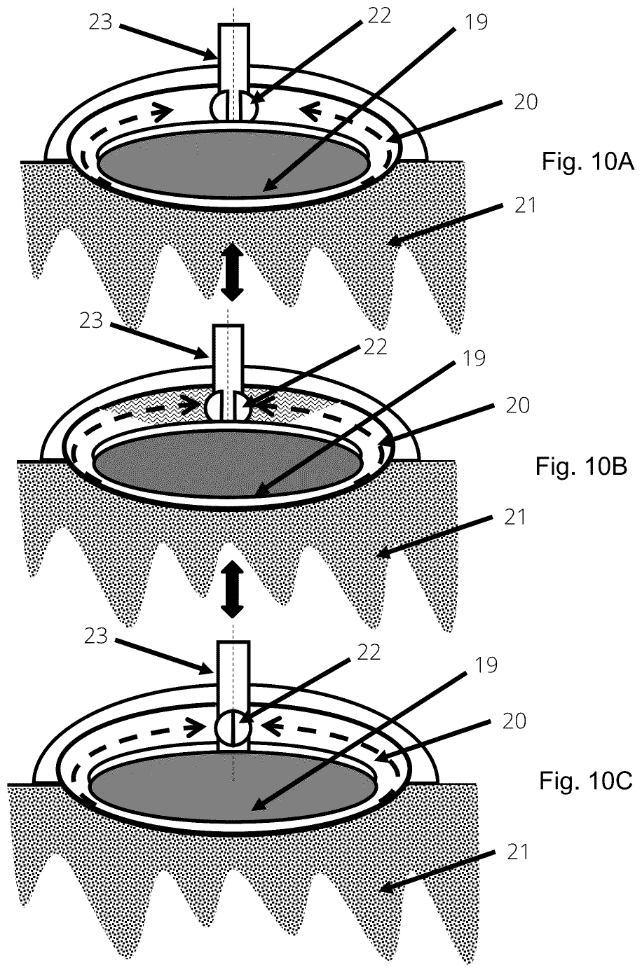

FIGS. 10A-C are schematic diagrams of a heat pack showing reversible pore opening and closing, in accordance with some embodiments. For example, initially one or more pores 23 in a surface of a heat pack may be opened (e.g., by an air flow control) to provide oxygen for an exothermic chemical reaction. The exothermic chemical reaction in heat source 19 may heat the buffer 20 close to the skin 21 of the user. As the exothermic reaction progresses, buffer 20 may absorb more and more heat, and/or a phase transition from a first phase to a second phase may proceed through the phase changing material buffer. Once the temperature of buffer 20 has reached a predetermined temperature and/or all the phase changing material has transformed from a first phase to a second phase, then the air flow control 22 may be closed. Optionally, flow control is closed by the transformation the volumetric change of the PCM, caused by phase changing, to a mechanical movement. This may prevent and/or reduce air flow to heat source 19 thereby reducing the reaction rate of the exothermic chemical reaction, and reducing the heat produced. Optionally, this process may be reversible.

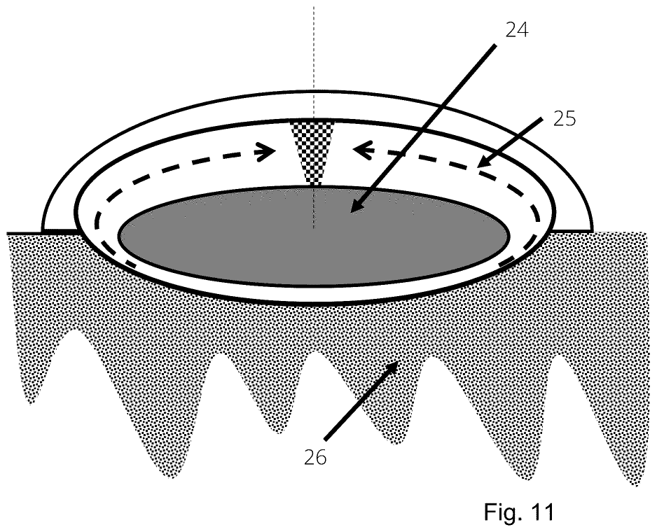

FIG. 11 is a schematic diagram of a heat pack, in accordance with some embodiments. For example, the heat produced by the heat source 24 may be absorbed by the body 26, and may therefore the amount of heat produced by the heat source may not exceed the phase change material's 25 heat capacity. Therefore, there may be no need for a feedback loop and/or air flow control.

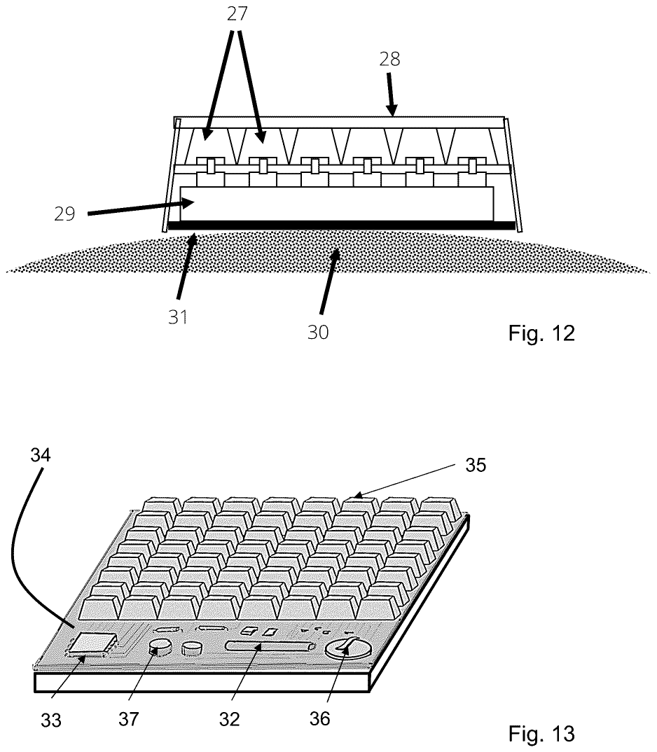

FIG. 12 is a schematic diagram of a cross-section view of a heat pack, in accordance with some embodiments. For example, the temperature of the heat pack may be controlled by controlling the rate of the chemical reaction by controlling the supply of fuel (e.g., the iron). Optionally, the heat pack may include one or more reaction capsules 27, which may be covered by a thermal isolation barrier 28. Optionally, the one or more reaction capsules 27 may be in fluidic contact with a buffer 29 which may include a phase changing material. The buffer 29 may be separated from the skin 30 of the user by a high thermal conductivity layer 31. Optionally, the one or more reaction capsules 27 may include one or more pores and/or passageways (not shown) for air to flow into the reaction capsules 27.

FIG. 13 is a schematic diagram of a perspective view of a heat pack, in accordance with some embodiments. According to some embodiments, the heat pack may include one or more sensors 32. Optionally, the sensors 32 may include heat sensors, e.g., thermocouples, etc. Optionally, the one or more sensors 32 may be connected to an integrated controller 33 (e.g., an integrated circuit and/or electronic controller). For example, an electronic controller 33 may respond to a temperature sensor 32. Optionally, when the heat pack is too cool the controller 33 may release material to fuel the chemical reaction. For example, if the exothermic chemical reaction is iron oxidation the controller may release portions of iron to be oxidized.

Optionally, the system may include a flexible circuit board (PCB) 34 to which may be attached one or more reaction capsules 35 including heating source material. The PCB 34 may include a controller 33, one or more temperature sensors 32, a power source 36 and a network connector (e.g., Bluetooth connector) 37. Optionally, the one or more sensors 32 may be connected to an integrated controller 33 (e.g., an integrated circuit and/or electronic controller). For example, an electronic controller may respond to a temperature sensor. Optionally, when the heat pack is too cool, the controller may release material to fuel the chemical reaction, and/or if the heat pack is too hot, the controller may cut off fuel supply to the chemical reaction. For example, if the exothermic chemical reaction is iron oxidation the controller may release and/or prevent release of portions of iron to be oxidized.

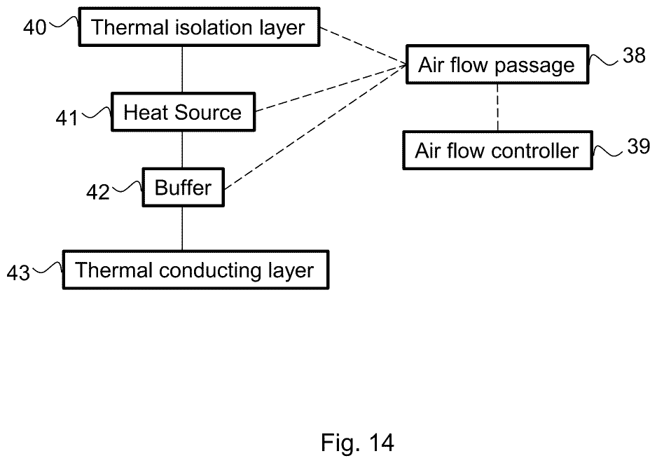

FIG. 14 is a block diagram of a heat pack, in accordance with some embodiments. For example, a heat pack may include a heat source 41 (e.g., fuel source), and a buffer 42 (e.g., a phase changing material), wherein the heat source 41 and/or the heat pack is at least partially covered by a thermal isolation layer 40. The buffer 42 and/or the heat pack may be separated from the skin of the user by a thermal conducting layer 43. Optionally, the thermal isolation layer 43 and/or the buffer 42 may include an air flow passage 38 e.g., pore which may be configured to facilitate air flow to the heat source 41. Optionally, the air flow passage 38 may include an air flow controller 39. Optionally, the air flow controller 39 may be configured to control the amount of air entering the heat source 41 via the air flow passage 38, thereby controlling the amount of oxygen available for reaction, and therefore the amount of heat produced by the heat source 41.

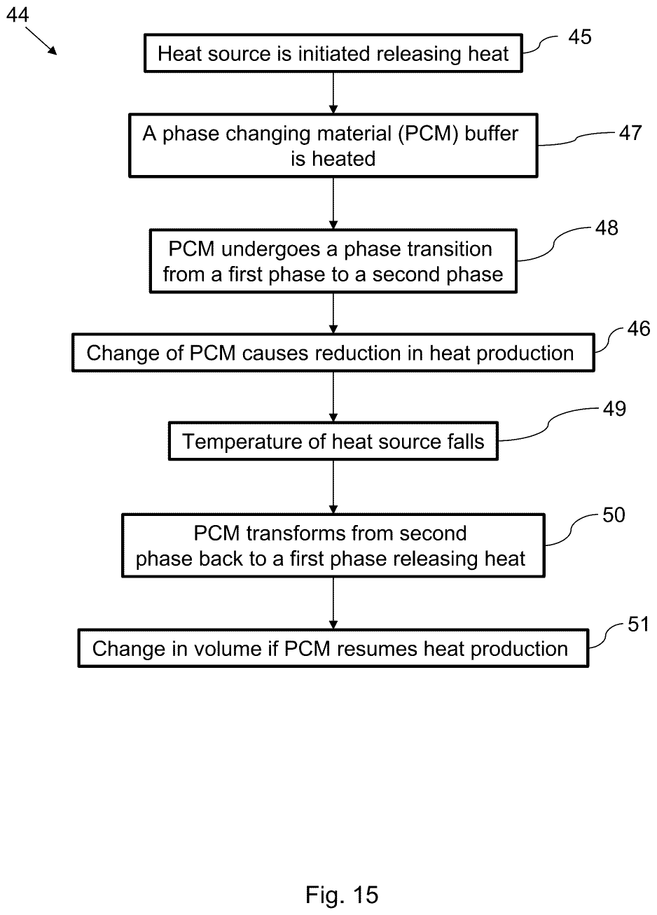

FIG. 15 is a flow chart of a method of using a heat pack, in accordance with some embodiments. For example, in method 44 a heat source is initiated 45 releasing heat. For example, the heat source may include an exothermic chemical reaction. The heat, is partially absorbed 47 by a phase changing material (PCM) buffer. The phase changing material buffer then undergoes (for example when a specific temperature is reached e.g., between 40 and 44 degrees C.) a phase transition 48 from a first phase to a second phase, absorbing 47 at least some of the heat from the heat source. Transition 48 of the PCM from a first phase to a second phase and absorbing 47 heat may facilitate the heat pack remaining in a desired temperature range (e.g., between 40 to 44 degrees C.). Optionally, the phase transition 48 of the PCM may cause a reduction 46 the rate of heat production. For example, the phase transition 48 may cause a volumetric change in the phase changing material. The change of volume may trigger a heat production reducing mechanism. For example, the change in volume of the PCM may limit a source of fuel and/or oxidant to an exothermic reaction. For example, the change in volume of the PCM may close a pore supplying air to an exothermic oxidation reaction. Reducing 46 the heat production, optionally cools 49 the heat source and/or the PCM. As the PCM cools it optionally goes through a phase transition 50 back to the first phase and/or releases heat. For example, releasing heat from the PCM optionally keeps the heat pack in the desired temperature range while the heat source cools 49. The phase transition 50 of the PCT back to the first change may then reverse the process that reduced 46 heat production and/or the heat source may resume 51 heat production. The negative feedback between the PCM and the heat source as the PCM transitions 48, 50 back and forth between the first phase and the second phase and/or the reaction rate is reduced 46 and/or resumed 51 may facilitate controlling the temperature of the heat pack.

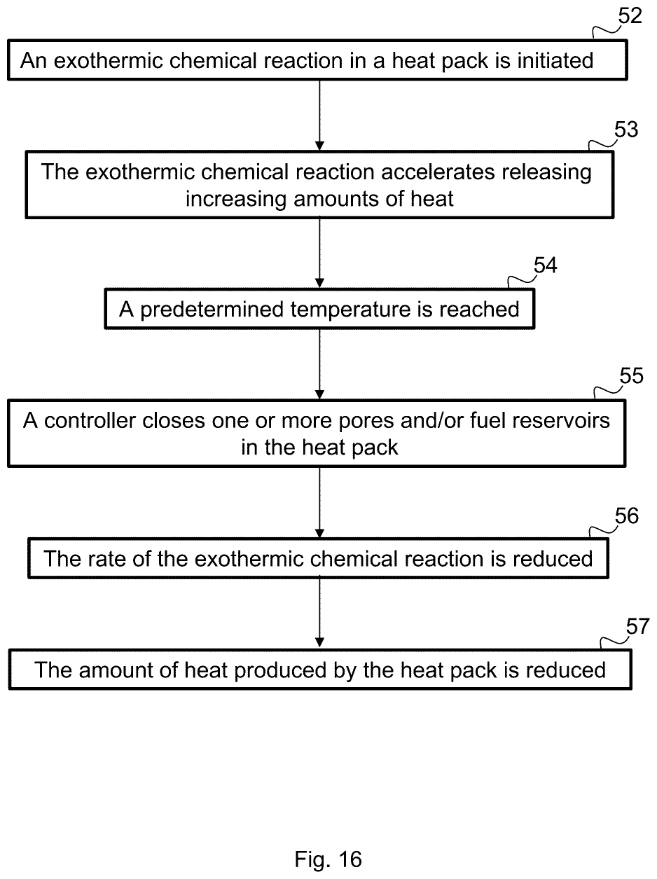

FIG. 16 is a flow chart of a method of using a heat pack, in accordance with some embodiments. For example, an exothermic chemical reaction in the heat pack is initiated 52. The exothermic chemical reaction accelerates 53 releasing increasing amounts of heat until a specific temperature is reached 54. A controller then closes 55 one or more pores and/or fuel reservoirs in the heat pack, reducing 56 the reaction rate of the exothermic chemical reaction, thereby reducing 57 the amount of heat produced by the heat pack.

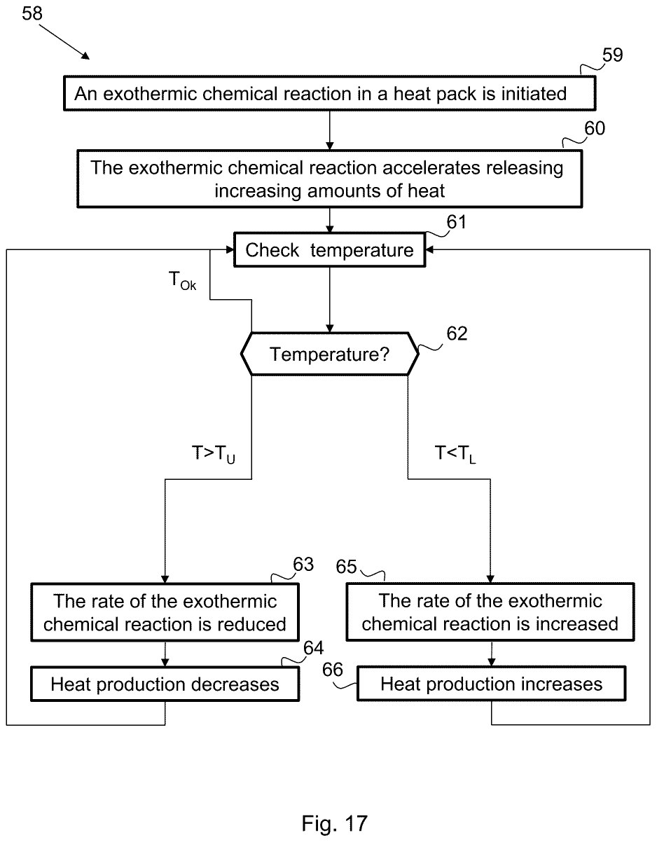

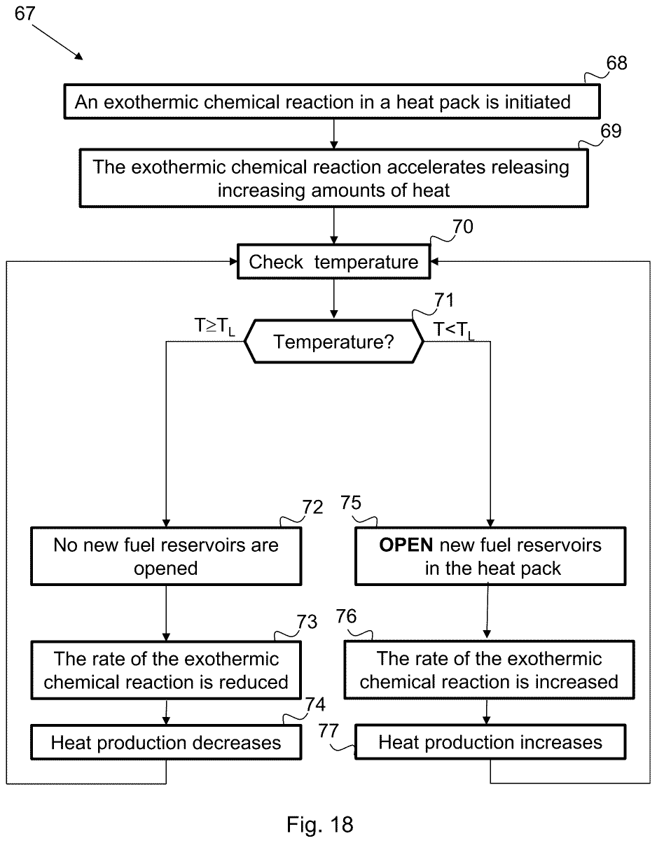

FIG. 17 and FIG. 18 are flow chart illustrations of methods of providing controlled heat, in accordance with some embodiments. Optionally, the methods may be combined.

FIG. 17 illustrates method 58 of controlling temperature (T) of a heat pack where a reaction rate can be increased or decreased. Optionally, the reaction rate may be controlled by opening and/or closing pores supplying air (e.g., O₂) to an exothermic (e.g., oxygenation) reaction. For example, in method an exothermic reaction in a heat pack may be initiated 59. The exothermic chemical reaction may accelerate 60 releasing increasing amounts of heat. The temperature of the heat pack may be checked 61 (e.g., by a sensor). When the temperature (T) 62 is below a lower threshold (T_{L}) one or more pores are opened, increasing the rate of the reaction 65 and/or increasing the heat production 66. When the temperature (T) 62 is above an upper threshold (T_{U}) one or more pores are closed, decreasing the rate of the reaction 63 and/or decreasing the heat production 64. When an optimal temperature (T) is reached (Tₒₖ) e.g., between a lower threshold (T_{L}) and an upper threshold (T_{U}), pores sizes are not changed, and the rate of the reaction remains stable (for example slowly being reduced as fuel is used up). Optionally pores may be controlled by an electronic controller and/or pores may be designed to be temperature sensitive without external control. In some embodiments, pores may be binary (either fully closed or fully opened). Alternatively, or additionally, pores may be progressive (may be fully or partially or completely closed). Optionally, once heat production has been reduced and/or increased, the temperature may be tested once more, and based on the new temperature reading one or more of the previous steps may be repeated.

The FIG. 18 mechanism is “one sided”, means no action is being done when overheating, but wait for decrease in temp since fuel is not being used. For example, in FIG. 12, 13 where a controller may increase a reaction rate, but once a new fuel cell is activated, it cannot be stopped and only stops slowly over time as fuel is reduced. For example, in method 67, an exothermic reaction in a heat pack may be initiated 68. The exothermic chemical reaction may accelerate 69 releasing increasing amounts of heat. The temperature of the heat pack may be checked 70 (e.g., by a sensor). When the temperature (T) 71 is below a lower threshold (T_{L}) one or more new fuel reservoirs are opened 75, increasing the rate of the reaction 76 and/or increasing the heat production 77. When the temperature (T) 71 is above an upper threshold (T_{U}) no new fuel reservoirs are opened 72, decreasing the rate of the reaction 73 and/or decreasing the heat production 74.

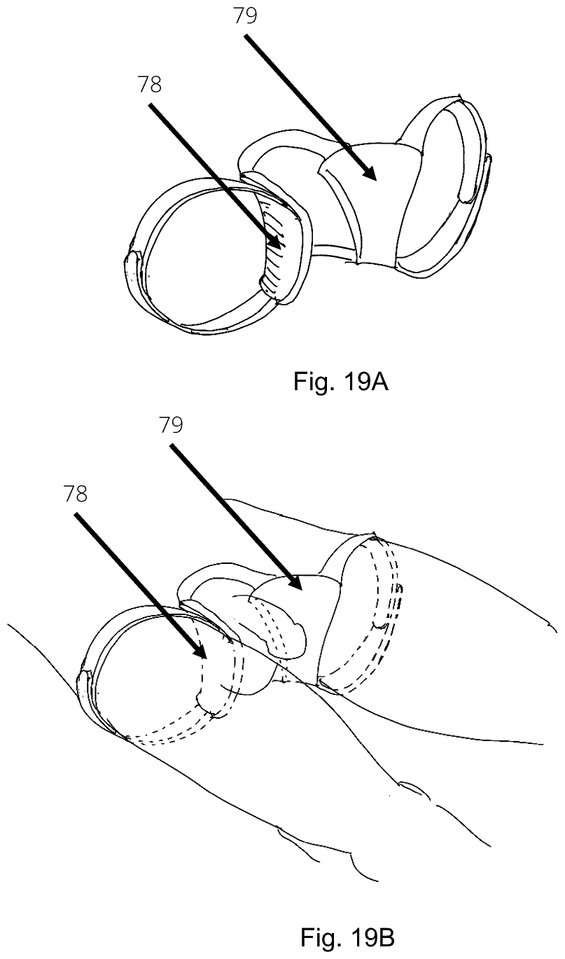

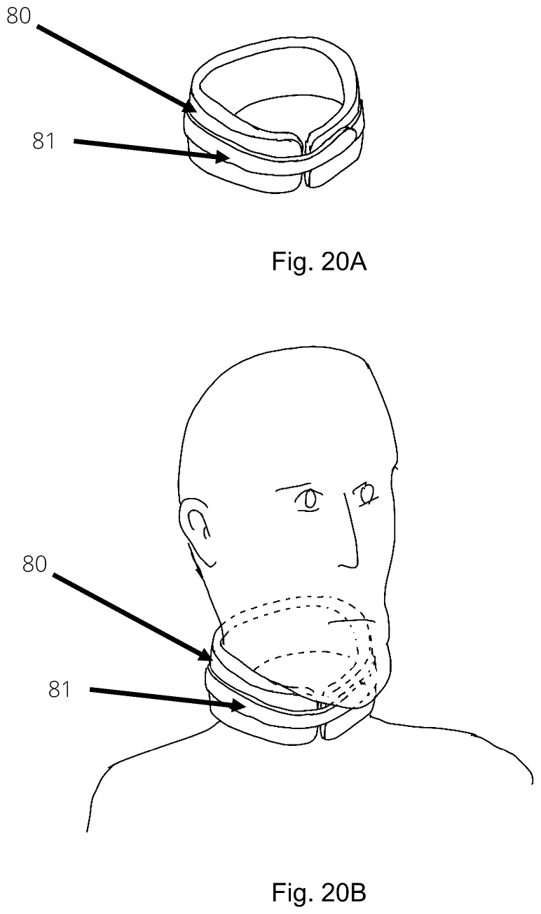

FIGS. 19A-B and FIGS. 20A-B are schematic diagrams illustrating anatomic design of a heat pack, and use thereof, in accordance with some embodiments. For example, the heat pack may be designed to fit the body. For example, a wearable harness may hold the heat-pack to the body and/or the heat-pack may be built into a wearable harness. Optionally, the heat pack and/or harness may have a variety of shapes, e.g., shaped to fit specific areas of a user's body. Optionally, the heat pack and/or harness may have a variety of sizes, e.g., to fit various ages and/or body stapes and/or body parts (e.g., parts of the body with large blood flow that can transfer large amounts of heat to the rest of the body e.g. neck, chest, groin, etc.). Optionally, the heat pack may include one or more support pads and/or straps, optionally, the support pads and/or straps may be configured to hold the heat pack to a user's body. For example, the heat pack may be a wide pad for lower back and/or chest and/or a thin and/or long pad for neck and/or a heat pack and/or support to hold to the groin (e.g., to the inner thigh). etc. In each case the heat pack may be shaped to fit the body and/or may be flexible enough to allow it to follow the contours of the body. For example, a heat pack 79 and/or support 78 may be configured for use in the groin (e.g., FIGS. 19A-B). For example, a heat pack 80 and/or strap 81 may be configured for use around the neck, arm, leg, etc. (e.g., FIGS. 20A-B).

FIG. 21 is a block diagram illustrating a heat pack, in accordance with some embodiments. For example, the system may include a heat source 84, a buffer 83, and a high conductivity layer 85. Optionally, the high conductivity layer may be a thermally conducting layer. Optionally, the high conductivity layer 85 may include a high conductivity pathway between the heat source 84 and the buffer 83 and/or the heat source and a contact surface with the subject. For example, high conductivity pathways may be located within one or more stitch lines or zones. Optionally, the high conductivity material may be a thermally conducting material. Optionally, stitch lines may directly connect between the heat source 84 and the user's body. Optionally, the stitch lines may transfer heat rapidly to the user's body. Optionally, the stitch lines may be backed by a phase changing material (buffer) 83. Optionally, with the stitch lines, when the heat source 84 begins to heat up, the stitch lines may facilitate rapid heat transfer from the heat source 84 to the high conduction material 85 and/or adhesive region and/or to the body. For example, while buffer 83 remains cool, heat is conducted along the stitch lines from the heat source 84 to the stitch lines at the skin interface.

According to some embodiments, the high conductivity layer 85 may be small particles of a high thermally conducting material within buffer 83 (phase changing material). Optionally, the high conductivity layer 85 may include a metallic matrix within buffer 83. Optionally, the metallic matrix may be a net and/or screen and/or mesh and/or metal foam. Optionally, the high conductivity layer 85 may facilitate faster temperature rise and/or stable temperature during user heating. Optionally, the high conductivity layer 85 may increase heat conductivity, efficiency and/or work more rapidly.

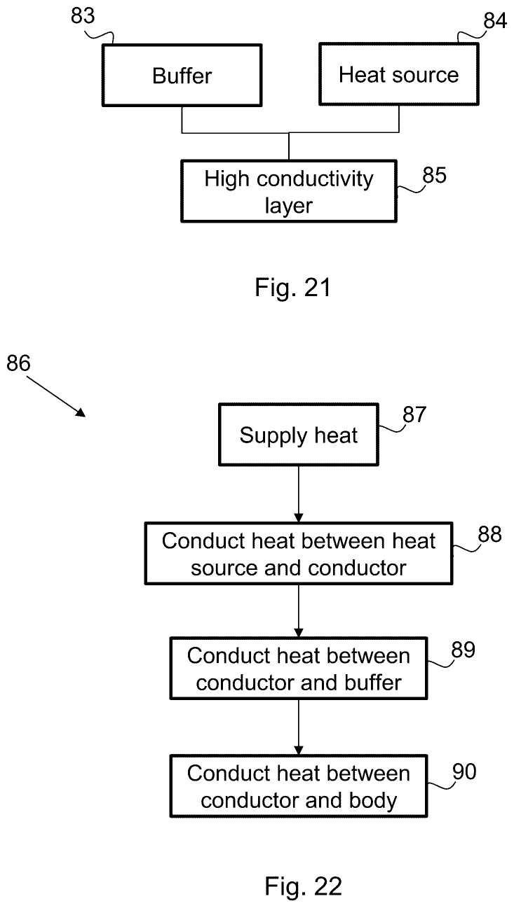

FIG. 22 is a flow chart for controlling a temperature of an outer surface of a heat pack, in accordance with some embodiments. For example, in method 86, supplying 87 heat from a heat source (e.g., an exothermic reaction), absorbing 88 heat to a highly thermally conductive material. Absorbing 89 a first portion of the heat from the highly thermally conductive material to a buffer. Optionally, the buffer may include a phase changing material configured to undergo a reversible endothermic phase transition as a temperature rises above a pre-defined temperature and/or a reversible exothermic phase transition as the temperature is lowered below the pre-defined temperature. Additionally, or alternatively, conducting 90 a second portion of the heat from the heat source along said highly thermally conductive material to an outer surface of the heat pack. Optionally, the first portion of said heat may be greater than, and/or similar to, and/or less than the second portion of the heat from the heat source.

These embodiments are provided by way of example and are in no means intended to limit the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

### GENERAL

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein, the terms “multiple” and “multi” are used interchangeably, and mean one or more, e.g., 1, 2, 3, 4, 5, 10, 20, etc.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A heat pack comprising:
a heat source comprising an exothermic reaction;
a buffer comprising a phase transition material; and
a thermally conducting material configured to conduct heat between the heat source to the buffer and an outer contact surface of the heat pack.

2. The heat pack according to claim 1, wherein the exothermic reaction comprises an iron oxidation reaction.

3. The heat pack according to claim 1, wherein the phase changing material undergoes a phase transition at a temperature ranging between 40° C. to about 45° C.

4. The heat pack according to claim 1, wherein the phase transition is reversible.

5. The heat pack of claim 1, wherein the thermally conducting material is configured to provide a path along which heat from the heat source is conducted directly to a body of a user.

6. The heat pack according to claim 1, wherein the thermally conducting material includes a thermally conducting layer.

7. The heat pack of claim 6, wherein the thermally conducting layer forms an interface between the heat source and the buffer.

8. The heat pack of claim 6, wherein the thermally conducting layer is configured to facilitate rapid transfer of heat to an outer surface of the heat pack while sharing the heat with the buffer.

9. The heat pack according to claim 1, wherein the buffer further comprises a metallic matrix of high thermal conductivity.

10. The heat pack according to claim 1, wherein the thermally conducting material is located within the buffer, the heat source, surface stitch lines, or a combination thereof.

11. The heat pack according to one of claim 1, wherein the thermally conducting material is located adjacent to the buffer, the heat source, surface stitch lines, or a combination thereof.

12. The heat pack according to claim 10, wherein the stitch lines are configured to contact a body of a user.

13. The heat pack according to claim 1, further comprising an insulating layer, wherein the insulating layer at least partially encloses the heat source.

14. A method for controlling a temperature of an outer surface of a heat pack, the method comprising:
supplying heat with a heat source;
absorbing said heat to a highly thermally conductive material;
absorbing a first portion of the heat from said highly thermally conductive material to a buffer comprising a phase changing material configured to undergo an endothermic phase transition at a pre-defined temperature; and
conducting a second portion of said heat along said highly thermally conductive material to an outer surface of the heat pack.

15. The method according to claim 14, wherein said highly thermally conductive material provides a path along which heat from the heat source is conducted directly to a body of a user.

16. The method according to claim 14, wherein said highly thermally conductive material provides a path along which heat from the heat source is conducted directly to an outer surface of the heat pack.

17. The method of claim 14, further comprising controlling a rate of an exothermic reaction of the heat source.

18. The heat pack of claim 1, further comprising:
A wearable harness configured for holding the heat pack to a body of the patient.

19. The system of claim 18, wherein said harness is configured to hold the heat source to a groin of the body.
